(19) United States
(12) Patent Application Publication
(10) Pub. No.: US 2016/0166276 A1
(43) Pub. Date: Jun. 16, 2016

(54) Surgical Device

(71) Applicants: Haley Huang, Baltimore, MD (US); Barbara Kim, Baltimore, MD (US); Tom Catullo, Baltimore, MD (US); Esteban Urias, Baltimore, MD (US); Stephen Johannesson, Alberta (CA)

(72) Inventors: Haley Huang, Baltimore, MD (US); Barbara Kim, Baltimore, MD (US); Tom Catullo, Baltimore, MD (US); Esteban Urias, Baltimore, MD (US); Stephen Johannesson, Alberta (CA)

(21) Appl. No.: 14/698,851

(22) Filed: Apr. 29, 2015

Related U.S. Application Data
(60) Provisional application No. 62/090,342, filed on Dec. 10, 2014.

(51) Int. Cl. A61B 17/32; A61B 17/16; A61M 29/00

(52) U.S. Cl. CPC A61B 17/320092; A61M 29/00; A61B 17/1644

(57) ABSTRACT

A surgical device having a blade head with an inner surface facing an underside of vertebra. The device includes a first bend connecting a neck member to the blade head. The first bend includes a curvature that befits a geometry of the underside of the vertebra adjacent to an exposed area of a spinal canal. The curvature is measured between the blade head and the neck member. Moreover, the blade head includes a blade edge lengthwise. The blade head further includes a dull tip. The device also includes a stem connecting to the neck member.

### CROSS-REFERENCE TO RELATED APPLICATION

This is a nonprovisional of the provisional application, Ser. No. 62/090,342, filed on Dec. 10, 2014, whose disclosure is incorporated by reference in its entirety herein.

### FIELD OF THE INVENTION

Embodiments of the invention generally relate to a surgical device for decreasing the incidence rate of surgical injury during revision spinal surgeries.

### BACKGROUND

A cerebrospinal fluid (CSF) leak is a serious complication associated with spinal surgery, especially those involving laminectomies, in which cerebrospinal fluid escapes past the protective layer of tissue (the dura) surrounding the spinal cord through tears or punctures, and subsequently causes the pressure around the brain and spinal cord to drop.

Most CSF leaks occur during surgical procedures in the spine, and especially as a byproduct of revision surgeries. Because scar tissue forms as a result of primary surgeries and adheres to the dura, the dura becomes attached to the overlying bone and therefore increases the risk of a dural tear when the tissue is separated from the bone, as well as when the scar is separated from the dura. There is currently no device on the market that deals specifically with the separation of scar tissue, bone, and dura in the spinal area. Instead, doctors use a variety of instruments under the umbrella of periosteal elevators, which generally have a spatula-shaped end engineered to scrape tissue from the bone. However periosteal elevators are general surgical tools that are not designed to tear tissue gently, and can therefore easily damage the dura if the surgeon generates too much shear force or makes contact with the dura due to lack of vision.

Prior art such as ones shown in FIG. 1 and FIG. 2 include a curved ultrasonic blade with a trapezoidal cross-section.

Another prior art illustrates an ultrasonic device for cutting and coagulation, such as the one shown in FIGS. 3-5. This ultrasonic device has curved blades on both sides of the tip as well as the head of the tip.

A further prior art in FIGS. 6-7 shows an ultrasonic surgical blade with improved cutting and coagulation features.

### SUMMARY

Embodiments of the invention overcome the shortfalls of prior technologies by decreasing the amount of force necessary to remove this scar tissue from surrounding bone, greatly reducing the probability of tearing the dura as a result of the revision surgery. In one example, aspects of the invention differ from the prior art because they feature a full-body L-shaped curve. Additionally, unlike the angled head of the prior art, embodiments of the invention employ sharp sides along a tip terminating at a dulled distal end. Moreover, embodiments of the invention do not provide wide semicircle-angled side blades that extend all the way to the head of the tip and the front blade and differ from the spoon-shaped angle of this ultrasonic tip. Rather than a multi-faceted geometry and angled head with 8 sides, embodiments of the invention are designed with four sides and a blunt head unlike many of the prior art's angled head. Furthermore, the angle of the device of the embodiments of the invention is smaller than those of the prior art.

Embodiments of the invention provide at least the following features overcoming the shortfalls of the prior art. For example, it includes features such as a double-bent L-shape angle, a dull head, straight wedge blades on the sides, allowance for irrigation, and allowance for detachment. The L-shape angle is customized to the underside of the spinal canal so there is no room for the tip to penetrate the dura. In one embodiment, a blunt head was used in the design instead of a blade or angled head in order to decrease the risk of dural tear if the ultrasonic tip comes in contact with the dura. Finally, in another embodiment, the design has blades on both sides to allow the user to extend the initial downward incision to the sides.

Aspects of the invention are shaped to work specifically in the spinal region and require less force from the surgeon to separate and/or cut scar tissue. Combined, these features reduce the risk of damaging the spinal cord during surgery. Additionally, embodiments of the invention use a shape that ideally is configured to adapt to the ultrasonic technology, which is also utilized in spinal surgeries for various reasons. The advantage of these choices also provides the ease of adoption into the current medical standard of care.

High frequency vibrations concentrate force onto the edges of embodiments of the invention, which allows the separation and/or cutting of scar tissue with much less force applied by the surgeon. Without high frequency vibrations, a tip according to embodiments of the invention also has the advantage of allowing lateral separation and/or cutting while maintaining a degree of safety for the spinal cord underneath with a dulled tip. These advantages translate to less chance of spinal cord injury and reduced operating room time.

Moreover, the radii of curvature of the tip shaft employed in embodiments of the invention, as well as the dimensions of the sharp edges of the tip itself may be changed as needed. For example, the connection point of the tip and the rest of the surgical device may be changed and replaced as needed, and connections may be determined by the rest of the surgical device.

### BRIEF DESCRIPTION OF THE DRAWINGS

FIGS. 1-7 illustrate one or more prior art of the invention.

FIG. 8 illustrates a perspective view according to one embodiment of the invention.

FIG. 9 illustrates another perspective according to one embodiment of the invention.

FIGS. 10A to 10C illustrate different positions of one end of an irrigation opening according to one embodiment of the invention.

FIG. 11 illustrates a perspective view of a surgical device in operation according to one embodiment of the invention.

FIG. 12 illustrates a side view of a surgical device in operation according to one embodiment of the invention.

FIGS. 13A to 13D illustrate perspective views of a first bend of a surgical device according to one embodiment of the invention.

FIGS. 14A to 14C illustrate perspective views of a second embodiment of a first bend of a surgical device according to one embodiment of the invention.

FIG. 15 illustrates the other end of an irrigation opening according to one embodiment of the invention.

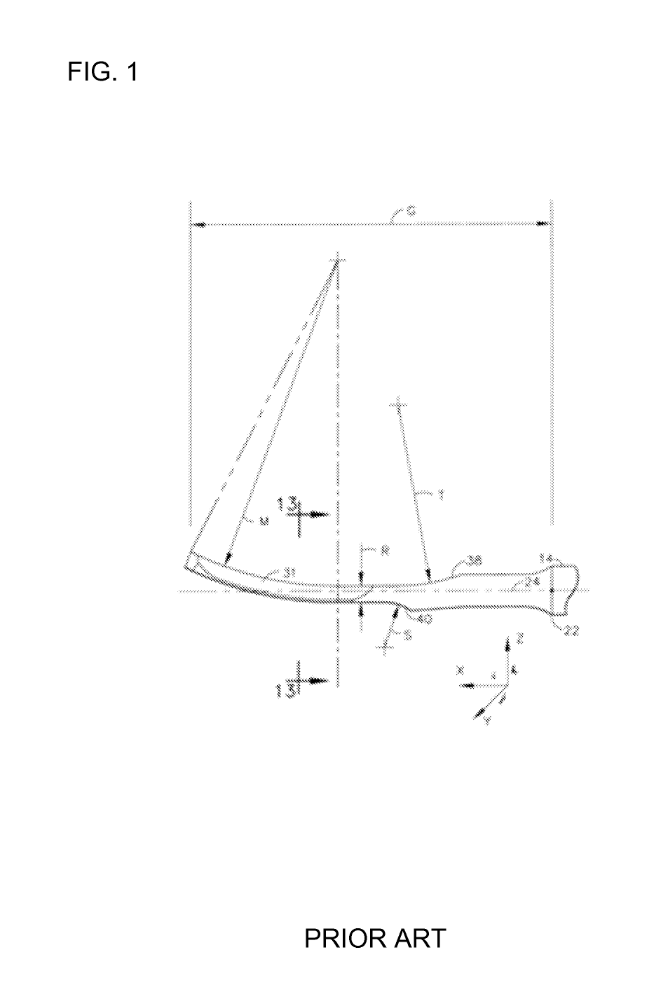

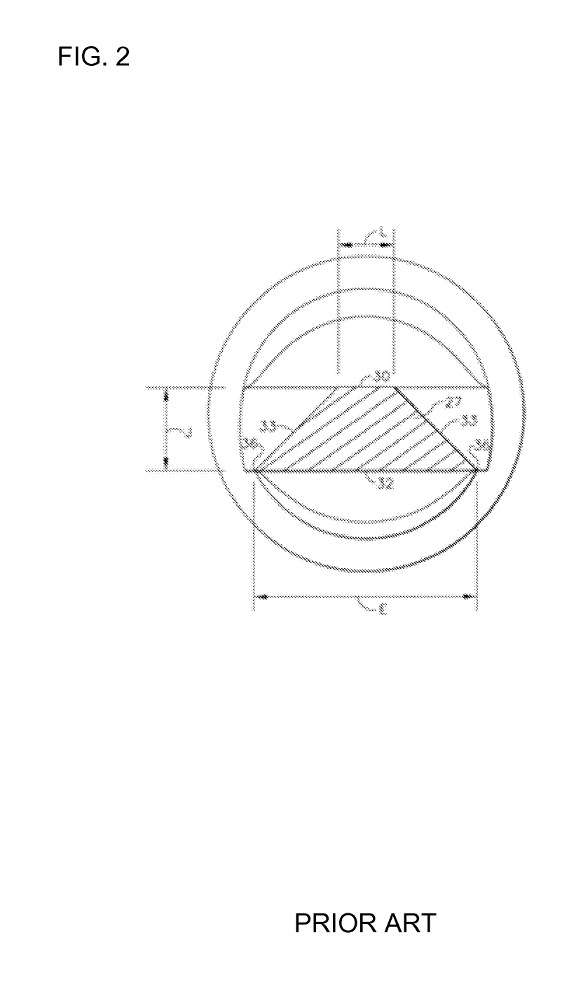

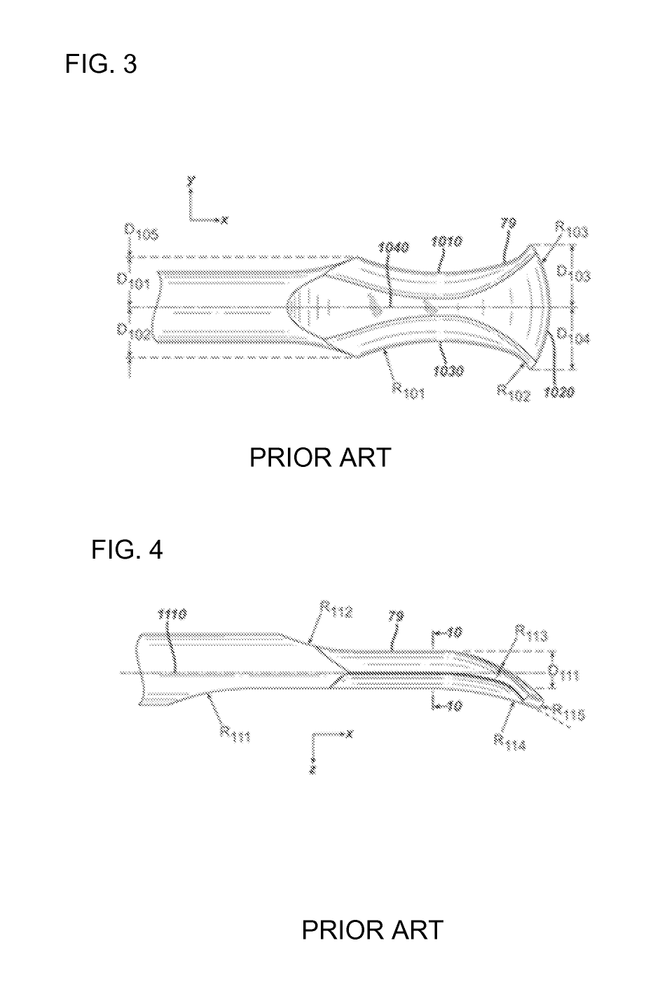

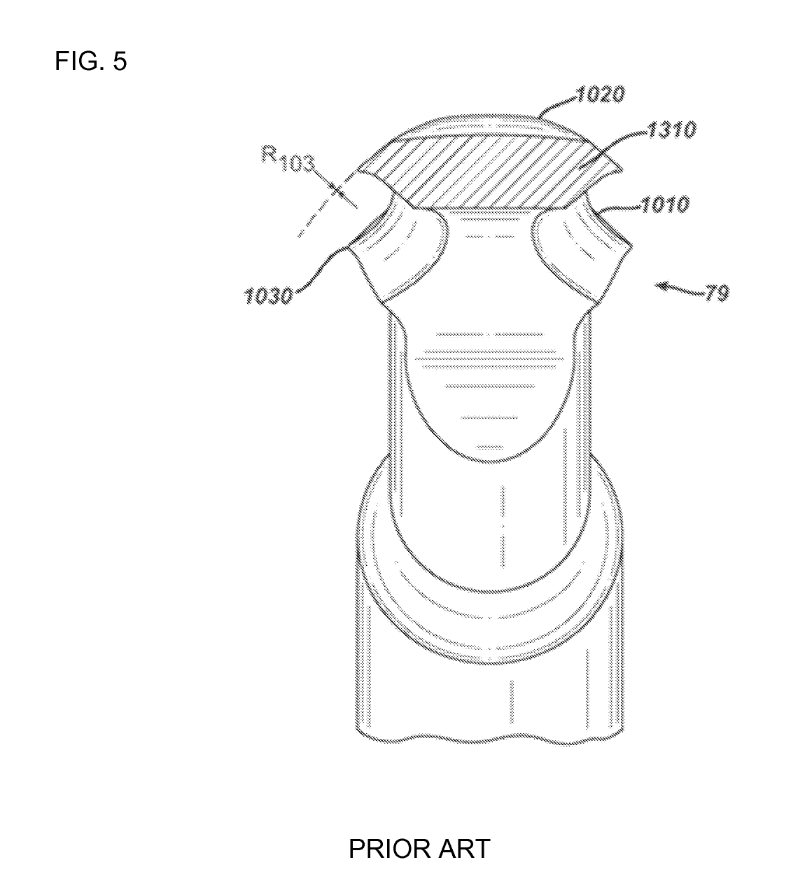

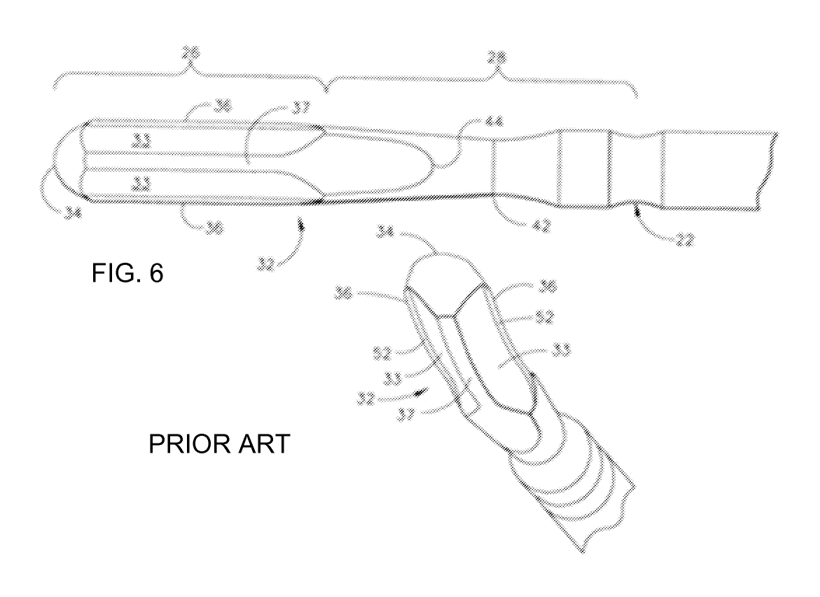

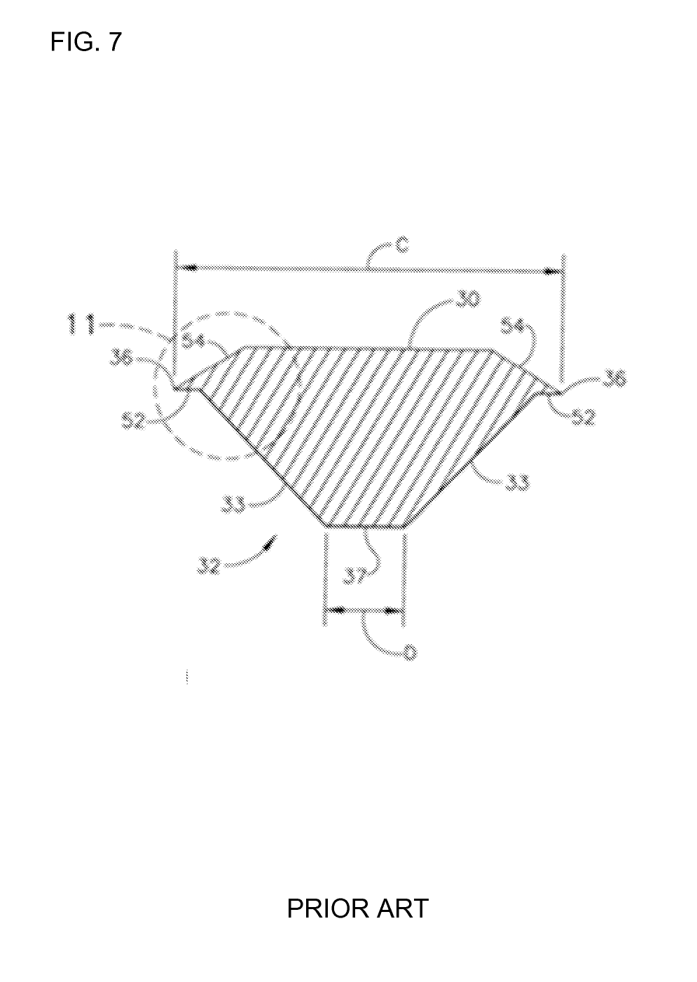

Corresponding reference characters indicate corresponding parts throughout the drawings.

### DETAILED DESCRIPTION

There are currently no surgical tools on the market specifically designed to separate and/or cut through scar tissue in the spinal area during spinal surgery. Instead, surgeons use generalized tools that require excessive force to perform this function. However, excessive force in the spinal area poses a severe safety hazard, often leading to tears in the dura mater, or damage to the spinal cord.

Aspects of the invention detailed herein include a modified tip for any surgical device that is designed to separate and/or cut scar tissue effectively in the spinal area while reducing the probability of injuring the spinal cord if the tip comes into contact with it. The tip design features a L-shaped curvature that befits the geometry of the spine, sharp edges on either side of the tip that enable it to cut scar tissue, and a dull tip that offers protection to the spinal cord underneath. The tip's tissue-penetrating capacity is optimized when attached to a handle piece that generates amplified ultrasonic vibrations, although the tip can also function as an addition to any other surgical device meant for the spinal area. In the case ultrasonic vibrations are utilized, the tip also offers several different means of cooling through specifically placed irrigation openings.

As part of a design project, many clinicians around the country were surveyed for possible improvements that could be made to current tools used in operating rooms. Over several months, clinical data and surgeon responses were gathered, which identified the need for a surgical tool that can more easily separate and/or cut scar tissue during revision spinal surgeries.

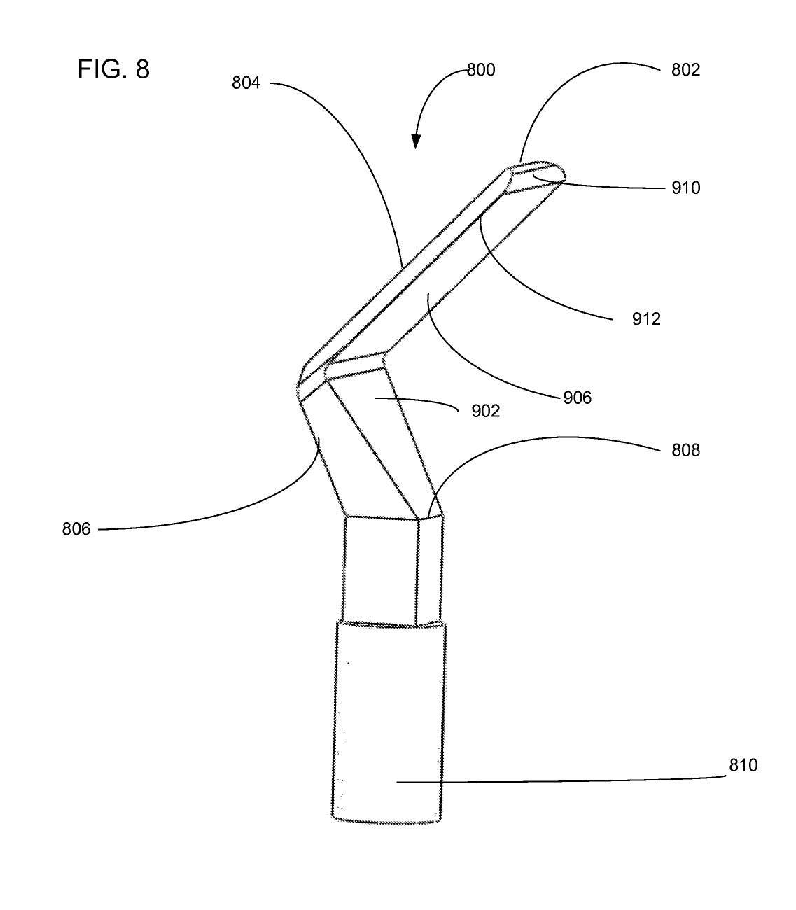

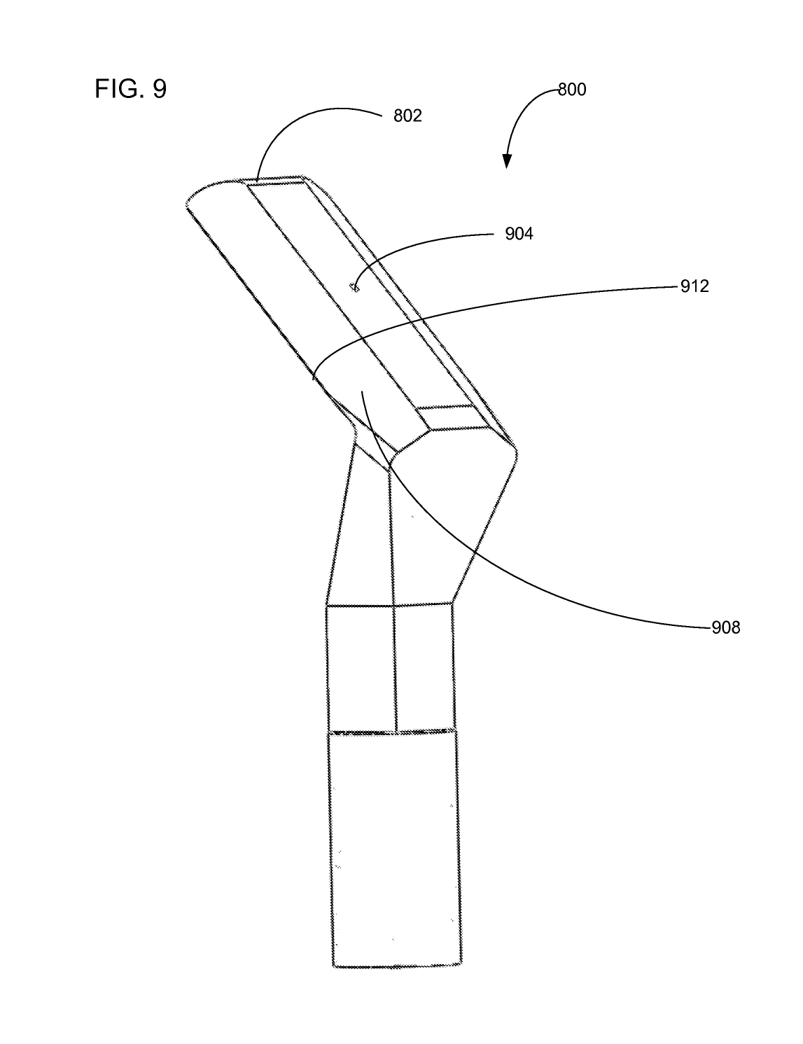

Referring now to FIGS. 8-9, perspective views illustrate a surgical device 800 according to one embodiment of the invention.

Referring to FIGS. 8-9, a device 800 may be used as the distal end of a surgical apparatus (not shown). In one embodiment, the device 800 may or need not be used with a curvature that befits a geometry of the vertebra adjacent with the exposed area of the spinal canal. In another embodiment, the device 800 may be made of materials such as titanium, aluminum, steel, stainless steel, and gold.

In one embodiment, the device 800 includes a blade head 802, a first bend 806, a second bend 808, and a stem 810. In another embodiment, the blade head 802 includes a tip 910, an outer surface 804, and an inner surface 906. The blade head 802 further includes a blade edge 912. The blade head 802 further includes a back surface 908 tapered from each of the blade edge 912 to the outer surface 804. In other words, the outer surface 804 of the blade head 802 includes the back surfaces 908 sloping laterally into the sharp blade edge 912 on both sides of the outer surface 804. In one example, the back surfaces 908 that slope to the sharp edge (e.g., the blade edge 912) allow for lateral separation and/or cutting that requires less force. As such, the inner surface 906 may be a flat or planar surface whose width is smaller than that of a length of the blade head 802. In one example, the length of the blade head 802 that allows for deeper penetration of the spinal canal may be used to reduce the need for vertical depression of the surgical device 800.

In one example, the blade edge 912 may exist one on side of the blade head 802. Alternatively, the blade edge 912 may exist on both sides of the blade head 802 along the length thereof. In an alternative embodiment, the blade head 802 may be as wide as its length. Also, the blade edge 912 may be incorporated into an L-shaped curved body that diverges into two or more wedge-shaped tips.

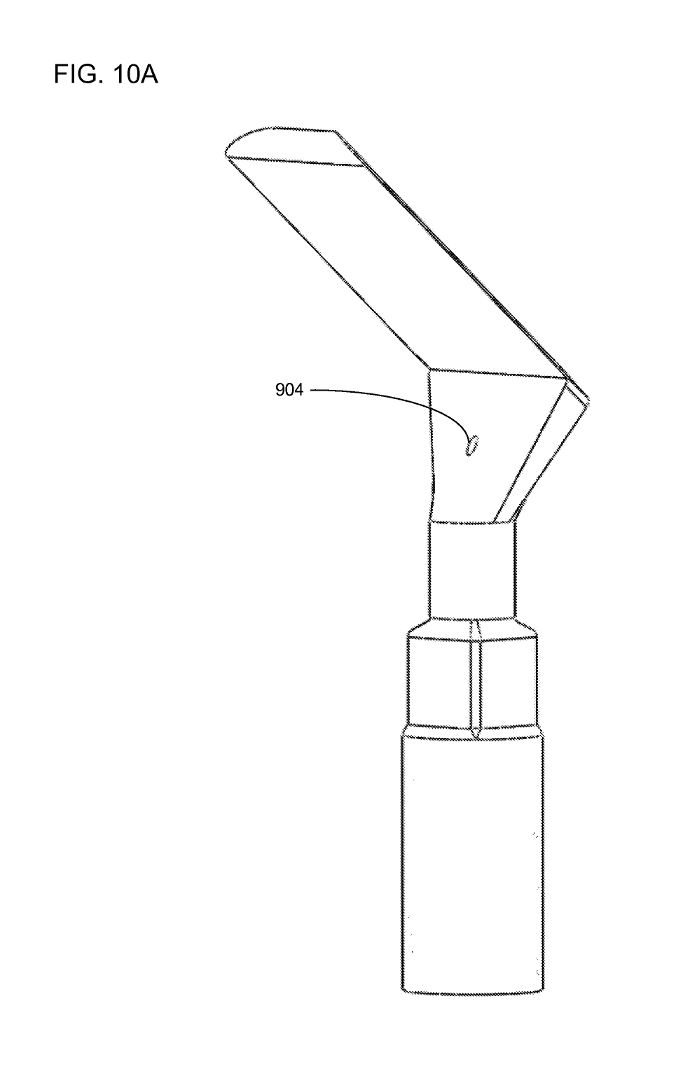

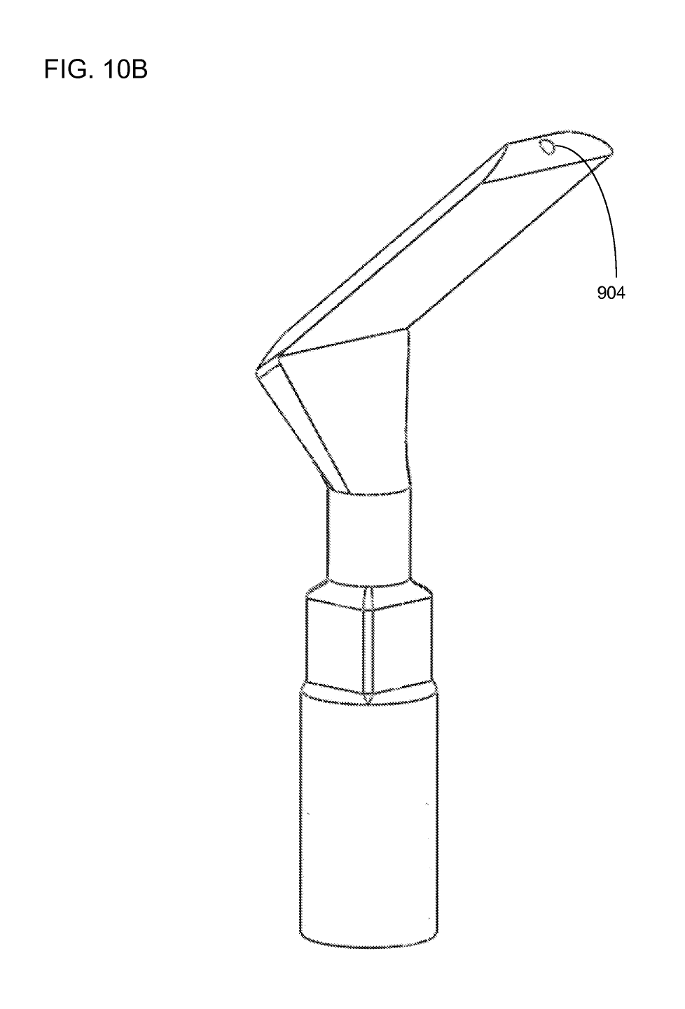

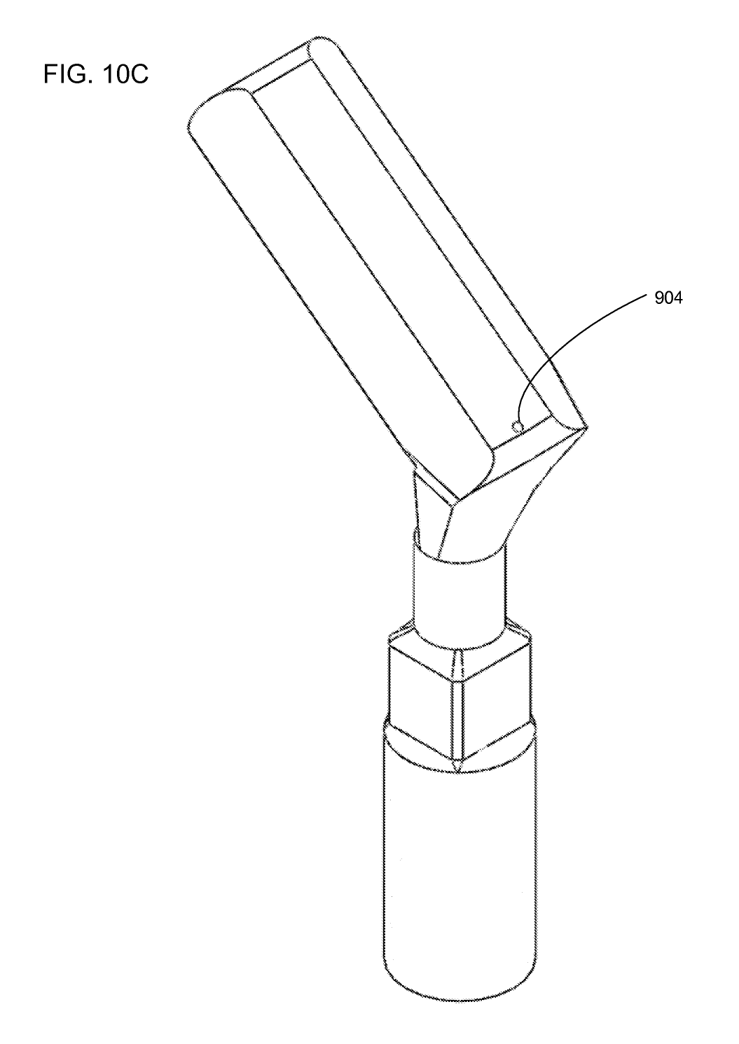

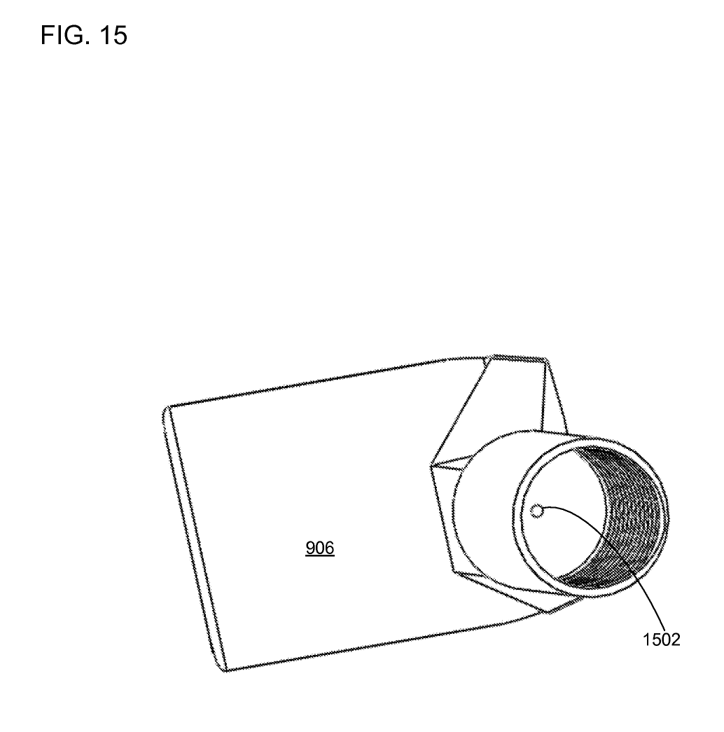

In a further embodiment, the second bend 808 connects the stem 810 and a neck member 902. The outer surface 908 may further include one end of an irrigation opening 904, as illustrated in FIG. 9. In one example, the one end of the irrigation opening 904 may be disposed at the neck member 902, as shown in FIG. 10A. In another example, one end of the irrigation opening 904 may be at or near the tip of the blade head 802, as shown in FIG. 10B. In another example, the one end of the irrigation opening 904 may be at or near the first bend 806, as shown in FIG. 10C. The one end of the irrigation opening 904 has an opening with a radius of about 0.30 mm, in one example. The other end, 1502, of the irrigation opening 904 may be disposed or exited in the stem 810 with a channel or passage connecting two ends of the irrigation opening 904 disposed in the internal part of the device 800 to guide, direct, or drain fluids to flow between the two ends of the irrigation opening 904, as shown in FIG. 15. In one embodiment, the irrigation opening 904 may be of different shapes or designs. For example, the irrigation opening 904 may be a slit, a hole, or a groove of a polygonal, circular, or other shapes without departing from the scope or spirit of the invention. In another embodiment, the irrigation opening 904 may be slightly smaller in diameter than the width of the blade head 802.

In one embodiment, the tip or a distal end 910 of the blade head 802 may be less sharp as the blade edges 912.

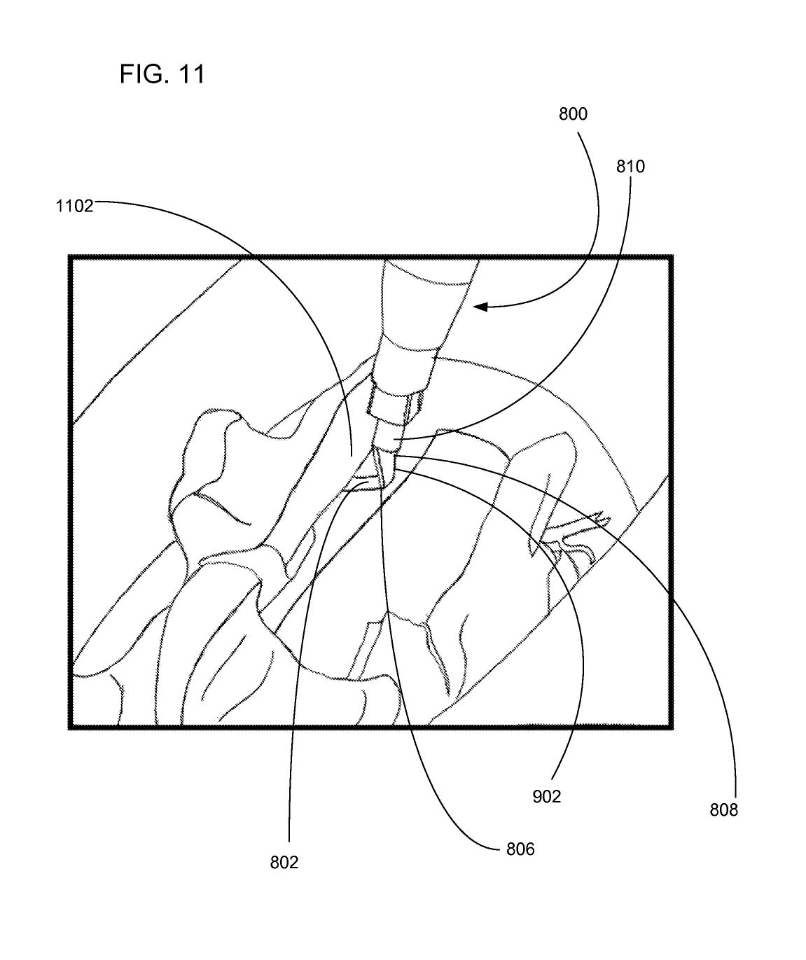

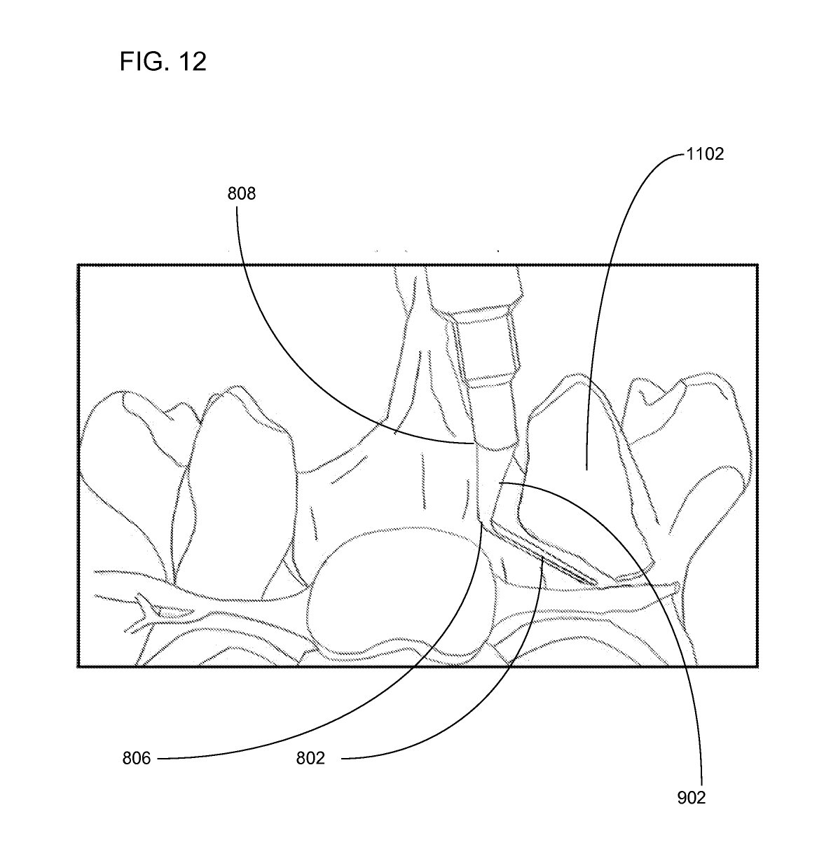

In one embodiment, the angle between the blade head 802 and the first bend 806 may be a value that, in conjunction with the angle between the first bend 806 and the second bend 808, extends the stem 810 at an angle of between 90 and 180 degrees in comparison to the plane of the blade head 802. For example, referring now to FIG. 11, a perspective view of a device 810 illustrates the device 810 in operation according to one embodiment of the invention. In this example, the inner surface 906 of the blade head 802 is facing a piece of vertebrate 1102. In the example, a surgeon (not shown) operating the device 800 may wish to use the device 800 to remove tissues on the vertebrate 1102 facing the blade head 802. As such, the surgeon would bring the blade head 802 to come in contact with the tissues. By gently moving the device 800 laterally and by resting the blade head 802 against the vertebrate 1102, the inner surface 906 of the blade head 802 with the blade edge 912 may be chipping or cutting the tissues away from the vertebrate 1102 as the surgeon gently alters the angle of the blade edge 912. See also FIG. 12 for a side view of the device 800 in operation according to one embodiment of the invention. As such, as depicted in FIG. 12, the first bend 806 may be designed to enable the inner surface 906 and the neck member 902 to surround, enclose, confine or corral the vertebrate 1102.

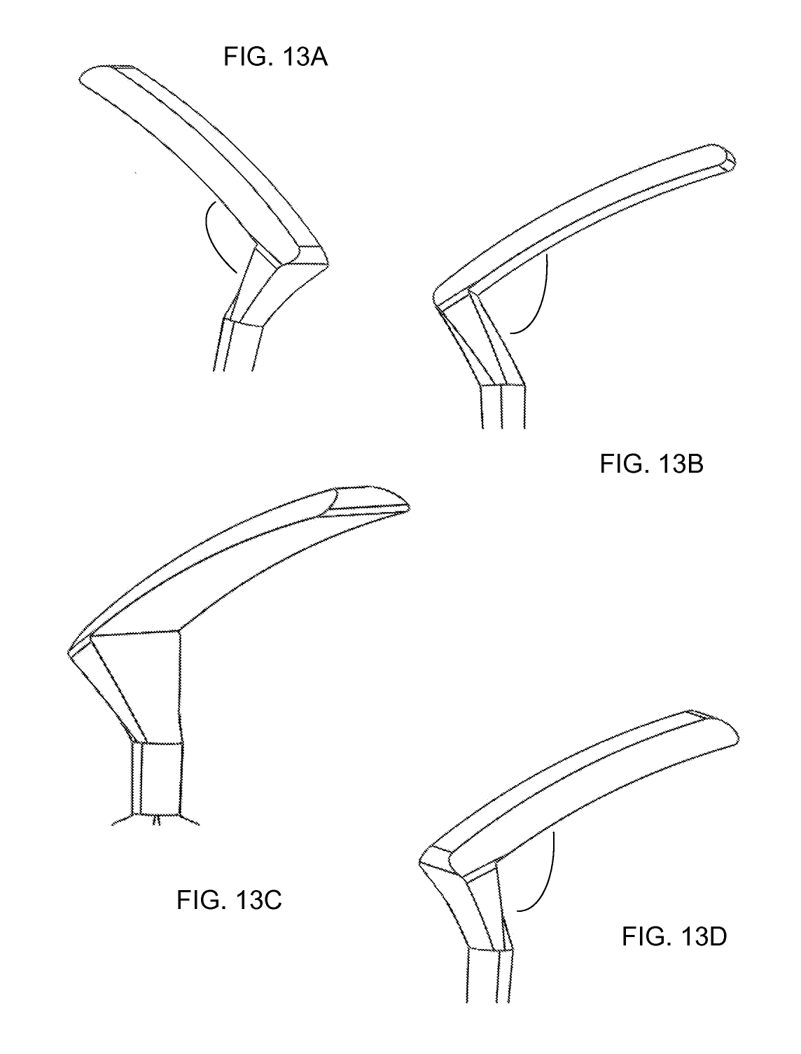

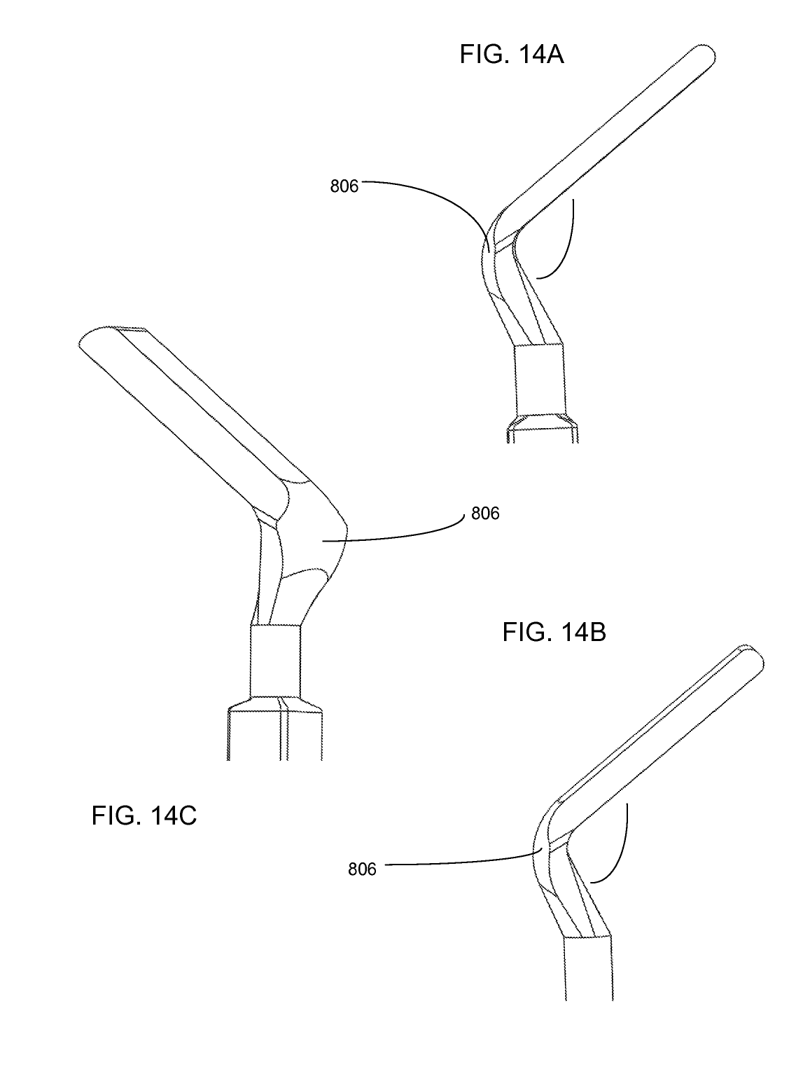

In one embodiment, the first bend 806 may be at least or about 75 degrees between the inner surface 906 and the neck member 902. In another example, the first bend 806 may be about 84 degrees. In a third example, the first bend 806 may be substantially 90 degrees between the inner surface 906 and the neck member 902. In another embodiment, the angle may be customized to have a curvature that befits the geometry of the vertebra adjacent with the exposed area of the spinal canal. FIGS. 13A to 13D and FIGS. 14A to 14C illustrate a number of different angles of the first bend 806 according to one embodiment of the invention.

In one embodiment, the blade head 802 has a width that is less than its length. In one example, length may be about 0.75 cm. In another example, the width may be about 0.5 cm. As shown in FIGS. 11 and 12, the length may be adjusted according to space limitation when operating on a subject. In another embodiment, the maximum length may be about 1.25 cm, as the length is limited by spinal canal space. In a further embodiment, the width may not exceed about 0.75 cm. In yet another embodiment, the length of the blade head 802 may be at least about 0.5 cm. In an alternative embodiment, the width of the blade head 802 may be at least about 0.25 cm. In a further aspect, the blade head 802 may be about 11.42 mm in length. Moreover, the neck member 902 may not exceed about 2.5 cm and may be at least about 1.25 cm.

In another embodiment, the blade head 802 may be about 0.99 mm in thickness. In a further embodiment, the inner surface 906 may be about 9.96 mm in length. It is to be understood that the inner surface 906 of the blade head 802 has direct contact with tissues or the vertebrate, such as illustrated in FIGS. 11 and 12. Hence, maximizing a surface area of the inner surface 906, while considering the limited amount of space in the spinal canal during surgery, is desirable.

In another embodiment, the second bend 808 has a maximum width as the blade head 802. In another embodiment, the neck member 902 connects between the first bend 806 and the second bend 808. In one example, as seen in FIGS. 11 and 12, the second bend 808 enables the surgeon to navigate the device 800 into the tight space of the surgical area. As such, the second bend 808 includes an angle between the stem 810 and the neck member 902 that is conducive to the navigation of the device 800 to the tight space. In a further embodiment, the second bend 808, the stem 810, and the neck member 902 may be integrated into one unit such that the angle of curvature of the second bend 808 is realized in the stem 810 or the neck member 902. In another embodiment, the second bend 808 may include a curvature that fits the geometry of the vertebrae to decrease the amount of vertical depression the device 800 may perform and decrease the tendency to implement downwards force when separating and/or cutting tissue.

In one aspect, the stem 810 may be about 7.4 mm in length. The stem 810 may further include a connector having about 2.55 mm in length. In a further embodiment, the stem 810 may be attachable, removable, or replaceable from a handle portion of an overall tool. For example, the stem 810 may be threaded, grooved, or having other structures to be configured to attach to another base instrument, such as an ultrasonic instrument. In this example, the base instrument may be connected to a console device that may include a microprocessor to execute a software program corresponding to generate high frequency ultrasonic vibration to the stem 810, such generating needed force to movements to the blade head 802 to remove tissues in the desirable location. In this example, it is to be understood that a plurality of software programs may provide computer-executable instructions or signals to the microprocessor to be executed in response to a user control or command.

In another embodiment, the stem 810 may be connected to another instrument. Alternatively, as the blade 912 of the blade head 802 becomes dull, the device 800 may be replaced. In such an embodiment, the stem 810 may include a bore or receptacle with grooves therein to receive a corresponding pin or plug of the base instrument such that the stem 810 is secured on the base instrument. In a further embodiment, the device 800 may be a standalone device with the stem 810 being a solid rod adapted for a surgery or a user to hold comfortably.

Embodiments of the invention may be implemented with computer-executable instructions, as related to the base instrument. The computer-executable instructions may be organized into one or more computer-executable components or modules. Aspects of the invention may be implemented with any number and organization of such components or modules. For example, aspects of the invention are not limited to the specific computer-executable instructions or the specific components or modules illustrated in the figures and described herein. Other embodiments of the invention may include different computer-executable instructions or components having more or less functionality than illustrated and described herein.

When introducing elements of aspects of the invention or the embodiments thereof, the articles “a,” “an,” “the,” and “said” are intended to mean that there are one or more of the elements. The terms “comprising,” “including,” and “having” are intended to be inclusive and mean that there may be additional elements other than the listed elements.

Having described aspects of the invention in detail, it will be apparent that modifications and variations are possible without departing from the scope of aspects of the invention as defined in the appended claims. As various changes could be made in the above constructions, products, and methods without departing from the scope of aspects of the invention, it is intended that all matter contained in the above description and shown in the accompanying drawings shall be interpreted as illustrative and not in a limiting sense.

We claim:

1. A surgical device comprising:
a blade head comprising an inner surface facing an underside of vertebra;
a first bend connecting a neck member to the blade head, said first bend comprising a curvature that befits a geometry of the underside of the vertebra adjacent to an exposed area of a spinal canal, said curvature being measured between the blade head and the neck member;
wherein the blade head comprises a blade edge lengthwise;
wherein the blade head comprises a dull tip; and
a stem connecting to the neck member.

2. The surgical device according to claim 1, wherein the blade head comprises an irrigation opening with one end thereof disposed at one of following locations: at a distal end of the blade head, at an inner surface of the blade head, at an outer surface of the blade head, at the first bend, or at the neck member.

3. The surgical device according to claim 2, wherein the one end of the irrigation opening comprises at least one of the following structures: a slit, a hole, or a groove of a polygonal or circular shape.

4. The surgical device according to claim 1, wherein the blade head is shaped to perform a type of surgery selected from the group consisting of spinal, neurological, and orthopedic surgery.

5. The surgical device according to claim 2, wherein another end of the irrigation opening exists at the stem.

6. The surgical device according to claim 1, wherein the first bend comprises an angle of about 75 degrees to 90 degrees between the blade head and the neck member.

7. The surgical device according to claim 1, further comprising a second bend connecting the stem to the neck member.

8. A surgical system comprising:
a surgical device comprising:
a blade head comprising an inner surface facing an underside of vertebra;
a first bend connecting a neck member to the blade head, said first bend comprising a curvature that befits a geometry of the underside of the vertebra adjacent to an exposed area of a spinal canal, said curvature being measured between the blade head and the neck member;
wherein the blade head comprises a blade edge lengthwise;
wherein the blade head comprises a dull tip; and
a stem connecting to the neck member; and
a console device connecting to the stem, said console device comprising a microprocessor to execute a software program corresponding to the surgical device, the microprocessor to provide a first control signal in response to the execution of the software program.

9. The surgical system according to claim 8, wherein the console device is configured to receive instructions from a user.

10. The surgical system according to claim 8, wherein the blade head comprises an irrigation opening with one end thereof disposed at one of following locations: at a distal end of the blade head, at an inner surface of the blade head, at an outer surface of the blade head, at the first bend, or at the neck member.

11. The surgical system according to claim 10, wherein the one end of the irrigation opening comprises at least one of the following structures: a slit, a hole, or a groove of a polygonal or circular shape.

12. The surgical system according to claim 10, wherein another end of the irrigation opening exists at the stem.

13. The surgical system according to claim 8, wherein the first bend comprises an angle of about 75 degrees to 90 degrees between the blade head and the neck member.

14. The surgical system according to claim 8, further comprising a second bend connecting the stem to the neck member.

15. An ultrasonic instrument system comprising:
a blade head comprising an inner surface facing an underside of vertebra;
a first bend connecting a neck member to the blade head, said first bend comprising a curvature that befits a geometry of the underside of the vertebra adjacent to an exposed area of a spinal canal, said curvature being measured between the blade head and the neck member;
wherein the blade head comprises a blade edge lengthwise;
wherein the blade head comprises a dull tip;
a stem connecting to the neck member; and
an ultrasonic vibration generator connected to the stem.

16. The ultrasonic instrument system according to claim 15, wherein the blade head comprises an irrigation opening with one end thereof disposed at one of following locations: at a distal end of the blade head, at an inner surface of the blade head, at an outer surface of the blade head, at the first bend, or at the neck member.

17. The ultrasonic instrument system according to claim 16, wherein the one end of the irrigation opening comprises at least one of the following structures: a slit, a hole, or a groove of a polygonal or circular shape.

18. The ultrasonic instrument system according to claim 16, wherein another end of the irrigation opening exists at the stem.

19. The ultrasonic instrument system according to claim 15, wherein the first bend comprises an angle of about 75 degrees to 90 degrees between the blade head and the neck member.

20. The ultrasonic instrument system according to claim 15, further comprising a second bend connecting the stem to the neck member.
